(19) United States
(12) Patent Application Publication
(10) Pub. No.: US 2011/0196376 A1
(43) Pub. Date: Aug. 11, 2011

(54) OSTEO-NAVIGATION

(76) Inventor: Burak Ozgur, Irvine, CA (US)

(21) Appl. No.: 13/024,239

(22) Filed: Feb. 9, 2011

Related U.S. Application Data
(60) Provisional application No. 61/302,634, filed on Feb. 9, 2010.

(51) Int. Cl. A61B 17/16

(52) U.S. Cl. 606/80

(57) ABSTRACT

A method and device for osteo-navigation is provided. The method may include identifying a target bone structure, insert of an orthopedic instrument including an optical probe for analyzing a characteristic of the target bone structure into the target bone structure, and navigating the orthopedic instrument into the target bone structure along a selected path in response to a signal from the optical probe.

### CROSS-REFERENCE TO RELATED APPLICATIONS

This application claims priority to the U.S. Provisional Patent Application No. 61/302,634 filed on Feb. 9, 2010.

### BACKGROUND OF THE INVENTION

Current techniques for navigating instrumentation around and inserting into bone employ basic knowledge of anatomy, learned skills, and various imaging techniques. For example, placing spinal screws requires knowledge of anatomical landmarks and tactile differentiation. Because of the important structures around and inside bone, misalignment of the spinal screw risks adverse health effects and increased morbidity. Imaging techniques to assist in screw placement are generally expensive, complex, and increase radiation to the patient and surgical staff. Therefore, a system to assist in the visual placement of instruments in and around bones of a patient is desired.

A device for monitoring penetration into anatomical members is described by U.S. Pat. No. 7,580,743 to Bourlion et al. The Bourlion device uses an electrical impedance to determine the location of the device tip. However, such a device only identifies the location of the tip at present and does not foresee the tissue in front of the tip. Thus, the device may determine once the tip has been misplaced, but cannot indicate that the tip is approaching an undesirable area before it is actually contacted.

Optical coherence tomography (OCT) technology is generally described in various patents including U.S. Pat. No. 6,950,692; U.S. Pat. No. 6,608,684; U.S. Pat. No. 7,227,629; U.S. Pat. No. 7,242,826; U.S. Pat. No. 7,538,886; U.S. Pat. No. 7,728,985; U.S. Pat. No. 7,821,643; U.S. Pat. No. 6,992,726; U.S. Pat. No. 7,573,020; U.S. Pat. No. 6,903,854. OCT has typically been used to view sub-surface layers of soft tissue to identify attributes of the tissue. In this regard, a probe abuts a soft layer structure, such as a mole, tissue sample, or skin, light is projected into the sample, and the reflected light is used to identify a desired characteristic of the sample. All of the patents and publications as referenced herein are incorporated in their entirety into the present application.

### BRIEF SUMMARY OF THE INVENTION

Various embodiments provide an improved system employed to give a surgeon real-time feedback of the bony anatomy in order to more accurately place instrumentation in and around the bone. The system may utilize tools that have embedded technology that gives the user real-time feedback as he/she navigates the anatomy. In an exemplary embodiment, the technology may include intravascular ultrasound (IVUS) or optical coherence tomography (OCT). The system may provide real time images of the procedure including the anatomical area and navigated instrumentation. The system may use the technology signals to analyze various attributes of the procedure to provide real time data to assist the surgical staff including real time imaging of the procedure, sizing information of the chosen instrumentation, estimates of distances to various anatomical features, audio or visual feedback of “danger zones,” etc.

To this end, in an exemplary embodiment method of osteo-navigation is provided. The method may include identifying a target bone structure, insert an orthopedic instrument including an optical probe for analyzing a characteristic of the target bone structure into the target bone structure, and navigating the orthopedic instrument into the target bone structure along a selected path in response to a signal from the optical probe.

In another exemplary embodiment, the method may include placing a pedicle screw in a vertebrae of a patient. For this embodiment, a surgeon may expose a spine of the patient, identify and mark an entry site for the pedicle screw into the vertebrae, advance a bone drill including an optical probe into the vertebrae, detect a substructure of the vertebrae with the optical probe by optical coherence tomography (OCT) in a path of the advancing bone drill, and select a continued path of the bone drill based on the detected substructure of the vertebrae.

According to alternative embodiments, an orthopedic surgical device for creating a bore in bone for placing a pedicle screw is provided. The orthopedic surgical device may include a handle, a drill bit coupled to the handle capable of creating the bore in bone, an optical probe including a fiber along a longitudinal axis of the drill bit, capable of projecting optical radiation from a tip of the drill bit, receiving backscattered radiation from a substructure in a trajectory of the drill bit, and creating an image of the substructure based on the received backscattered radiation, and a display for the image of the substructure.

### BRIEF DESCRIPTION OF THE DRAWINGS

FIG. 1 illustrates an exemplary embodiment of an OCT system in a hand-held intraoperative tool to assist surgeons placing spinal instrumentation;

FIG. 2A illustrates a section of the vertebral column including two vertebrae;

FIG. 2B illustrates a cut away of a vertebrae and the possible locations of a pedicle screw; and

FIG. 3 illustrates a flow diagram of the method of navigating an orthopedic device using embodiments as described herein.

While the invention is subject to various modifications and alternative forms, specific embodiments thereof have been shown by way of example in the drawings and will herein be described in detail. It should be understood that this invention is not limited to the particular forms disclosed, but on the contrary, the intention is to cover all modifications, equivalents, and alternatives falling within the spirit and scope of the invention.

### DETAILED DESCRIPTION OF THE INVENTION

Current techniques for navigating and inserting instrumentation around and into bone employ basic knowledge of anatomy and learned skills. However, newer techniques exist for computerized navigation. Disclosed is a system that may be employed to give the surgeon real-time feedback of the bony anatomy in order to more accurately place instrumentation and size the instrumentation chosen. This system utilizes tools that may have embedded technology that gives the user real-time feedback as he/she navigates the anatomy. This technology could be by way of OCT, as described herein.

Optical Coherence Tomography (OCT) is a technique for obtaining sub-surface images of translucent or opaque materials at a resolution equivalent to a low-power microscope. OCT uses optical reflection from within tissue to provide cross-sectional images. OCT measures the reflection time delays by comparing the back-reflected light signal to a controlled references signal. To create a two-dimensional image, the optic beam is moved laterally across the surface and in-depth profiles are obtained at discrete points along the surface. By obtaining these profiles over a lateral distance, a two-dimensional, cross sectional image is constructed.

OCT can deliver much higher resolution because it is based on light and optics, rather than sound or radio frequency radiation. An optical beam is projected into the subject, and light is reflected from the layers and sub-surface artifacts as the beam penetrates. Most of this light is scattered on its way back to the surface. The scattered light has lost its original direction and therefore cannot be used for imaging, which is why scattering material such as tissue appears opaque to the human eye. However, a very small proportion of the reflected light is not scattered. It is this non-scattered light that is detected and used in OCT. This non-scattered light has the unique property that it is coherent, so that it can be detected in an OCT instrument using a device called an optical interferometer. Essentially, the interferometer is used to separate the useless, incoherent, scattered light from the valuable, coherent, non-scattered light that can be used to generate an image. It also provides the depth and intensity information from the light reflected from a sub-surface feature, enabling an image of it to be built up, rather like an echo-sounder.

OCT technology can be combined with existing neuromonitoring technology and applications to give ultimately more accurate instrumentation. OCT provides live sub-surface images at near-microscopic resolution. For example, OCT may provide images of tissue morphology at a far higher resolution (better than 10 μm) than is possible with other imaging modalities such as MRI, ultrasound, X-ray, and CT. OCT also may enable instant, direct imaging of tissue morphology without requiring preparation of the sample or subject. Also, using OCT does not involve ionizing radiation, and therefore, enables fast, safe, and easy use in an office, laboratory, or clinic. Exemplary embodiments as described herein are generally described in conjunction with spinal applications including pedicle screw placement. However, the application is not so limited and may be applied to other orthopedic applications involving imaging or navigating in and around any bony structure.

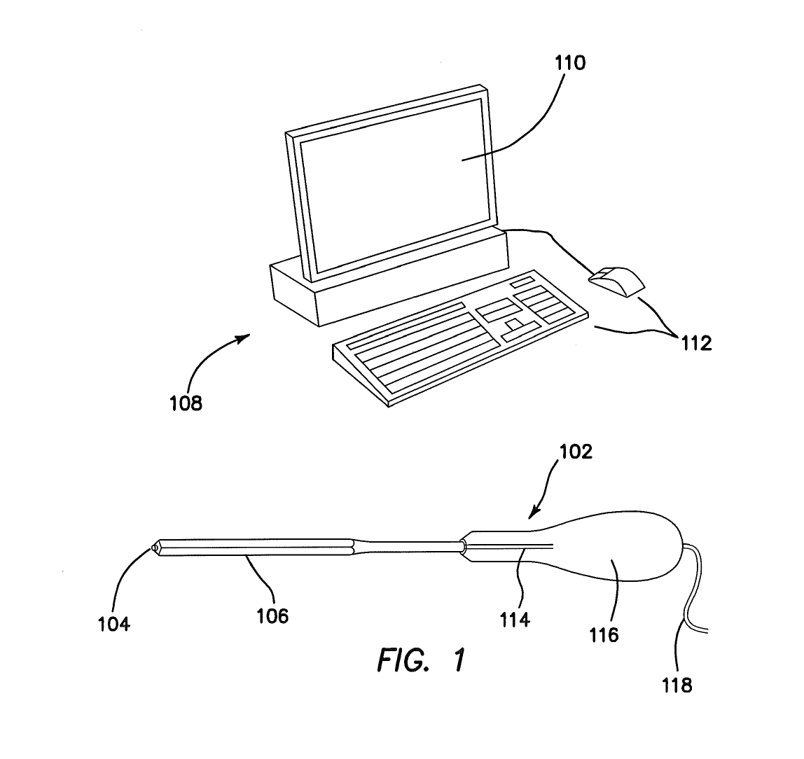

FIG. 1 illustrates an exemplary embodiment of an OCT system 100 in a hand-held intraoperative tool 102 to assist surgeons placing spinal instrumentation (for example, pedicle or vertebral body implantation of screws, plates, etc. . . ). The system may include the hand-held tool including an optical probe 104, spinal instrumentation 106, a console 108 including an interface 110, and other accessories such as a docking station (not shown). As illustrated in FIG. 1 the OCT system includes an optical probe 104 within the spinal instrumentation 106 of a bone drill. However, the optical probe 104 may be similarly applied with other orthopedic surgical devices, such as a bone awl, bone tap, screw driver, etc. or may be used as a standalone instrument inserted alongside, before, or after the orthopedic device. The console 108 may include peripheral devices 112 to assist in user input and output. The interface 110 may be a screen or other visual device for projecting an image produced from the optical probe 104, as well as other statistical information calculated by the console 108.

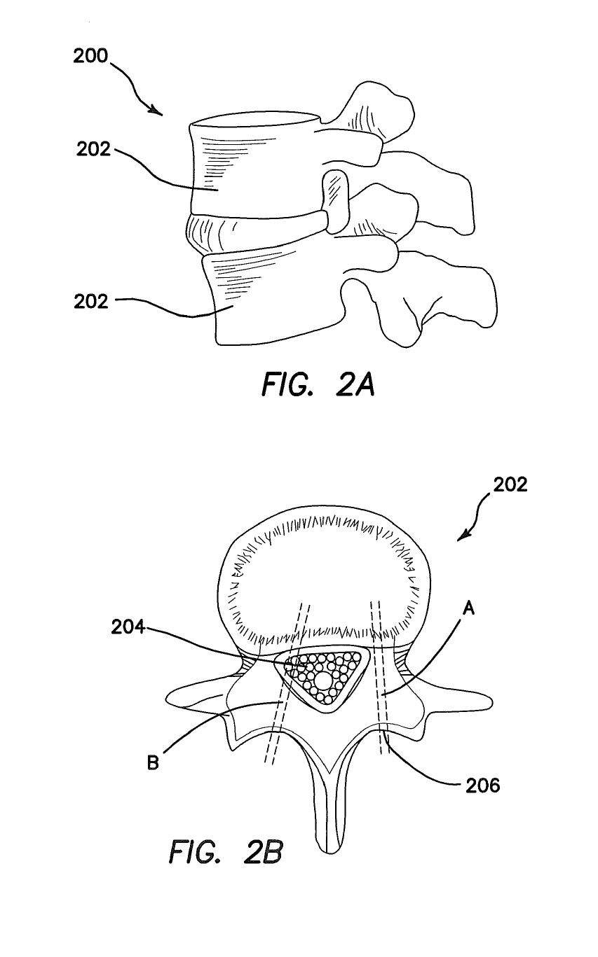

FIG. 2A illustrates a section of the vertebral column 200 including two vertebrae 202, separated by the intervertebral disc. FIG. 2B illustrates a cross sectional view of one of the vertebrae 202. FIG. 2B illustrates a possible position A (indicated by the dashed lines) of a bone screw, which is properly placed to avoid the soft tissue within the spinal canal 204. FIG. 2B also illustrates a possible position B (also indicated by dashed lines) of a bone screw, which is misplaced and penetrates the soft tissue 204. As seen in FIG. 2, the desired path to maintain the screw within the bony structure, while avoiding the soft tissue within the spinal canal is quite narrow. Properly placing the pedicle screw is demanding because of the close proximity of the spinal cord and the major blood vessels.

The hand-held tool 102 may be any orthopedic device used to navigate in or around bony structures. The hand-held tool may include a handle 116 and an instrumentation 106 end. As illustrated, the hand-held tool 102 instrumentation 106 includes a bone drill. However, the instrumentation end may be any orthopedic device. The hand-held tool 102 may also include a probe 104. The probe 104 may comprise of one or more optical fibers 114 and scanning mechanism (not shown). Light may be projected through a fiber 114 running through the device 102 into the tissue. Preferably, visible to near-infrared light is used for creating high resolution images. Reflected light from the tissue is captured by the fiber 114 and analyzed by the console 108. The optical fiber may be oscillated one-dimensionally to scan a tissue surface laterally. Multiples scans may be combined and displayed by the console interface 110.

The instrumentation 106 may include a drill tip, such as a bone drill, or other orthopedic device. The center of the device may be hollow to accommodate the probe 104 does a longitudinal axis of the tool 102. The instrumentation may be made of a material that does not optically interfere with the probe 104 such that the optical radiation from the probe is transmitted through the instrumentation and into the bone. Alternatively, the probe 104 may extend from a distal tip or lateral edge of the instrumentation so that the instrumentation is not in the path of the optical radiation from the probe. The tool may include a handle coupled to the instrumentation to assist in navigating the tool through the anatomical structures. The handle 116 may house the mechanical components and electronics, such as motors, gears, and circuitry to run the instrumentation and probe. Alternatively or in addition, some of the components may be external to the tool and coupled to the tool by a cord 118.

The projected and reflected near-infrared light is analyzed by the console 108 to produce an easy to use image of the scanned tissue on the console interface 110. The console may include imaging logic to produce an image from the collected reflected light and/or comparison logic to compare the produced image with a control image to determine a statistical variation. The imaging and comparison logic may be performed by hardware (circuitry, dedicated logic, state machines, etc.), software (such as is run on general purpose computer system or dedicated machine), or combinations of both. The imaging and comparison logic may be implemented with combination logic and finite state machines. The logic may include application specific integrate chip (ASIC), a field programmable gate array (FPGA), or processors, or any combination thereof Software may be used and may include machine instructions. Information may also be received from peripheral devices, such as a touch screen, mouse, keyboard, buttons, or other input/output devices. Information and images may be displayed on the peripheral devices, such as a screen, monitor, etc.

The console 108 may include a processor and memory that may be configured to store information and instructions for handling the imaging and comparison logic. The logic may include electrical circuits including, that allows information to be sent by and to the processor. Information may be sent to the processor by the optical fiber which captures reflected light from a desired tissue sample. Information may also be sent to the processor by peripheral devices to indicate desire system parameters. The processor may take the information received from the reflected light to create an image of the tissue sample. The created image may be displayed on a peripheral device to be viewed by a user. Information from the reflected light may also be compared to a control sample or image to determine a statistical variance. The memory may store instructions and/or information that allows the processor to calculate and determine the statistical variance of the created image from the control image.

The backscattered light may be captured by the optical fiber, and a signal corresponding to the optical intensity of the light delivered to the console. The console, using the intensity and other optical characteristics of the optical radiation backscattered by the anatomical structure and substructures, creates an image of the associated structures. The density and optical properties of the material under study may influence the backscattered light and may be used to analyze the material under study. For example, the exterior surface of the vertebrae, the cortical bone, is more dense than the inner cancellous bone within the vertebrae. Therefore, the image returned from the optical probe will be shallower for the cortical bone than the cancellous bone. The density variations between the various bone types may be detected by the intensity variations of the reflected signals and used by the console to determine the location of the probe tip. Therefore, once inside the bony structure, the console may detect the presence of a denser bone material and alert a use that the outer perimeter of the bone is approaching. Alternatively, a low density area may be detected in the probe path and used by the console to alert a user that the inner soft tissue of the spinal canal is within the probe trajectory. The system may indicate these variations by providing a visual image of the returned intensity signal as a representation of the detected biological material.

Alternatively or additionally, the system may provide other feedback to a user to assist in the navigation in and around the bony structure. For example, the console may use the known density and optical characteristics of the bone with the known optical radiation characteristics to calculate an actual distance to a transition from one substructure to another. Therefore, the system may alert a user of the actual distance to the end of the bone or to soft tissue within the bone. The system may provide other visual or audible indications when the probe is approaching one of the above described or other selected areas of interest, such as by a light indication, color indication, buzz, beep, audible decibel level, etc.

The probe may include one or more fibers for providing information of the material under study in more than one direction. For example, the device may include a central fiber that projects optical radiation along the longitudinal axis of the device from its distal end. This first fiber thus detects the anatomical features in the trajectory of the device distal tip. One or more additional fibers may also run along the length of the device and project separate optical radiation laterally from the device end. These signals may detect the anatomical features near the probe tip. One or more of the signals may be used to accurately locate the probe tip within the bony structure. For example, a first and second lateral signal may be used to detect the edge of the bone or the soft tissue. Once detected on either side, the surgeon knows that the proper path is taken and the probe is passing the narrow passage between the spinal canal and the bone edge. A longitudinal signal from the tip of the device may then be used to detect the cortical bone and ensure the device does not exit the bone on the opposite side from the entry location. The signals from the one or more fibers may be distinguished to reduce or prevent cross-scattering between the fibers by various methods. for example, cross-scatter may be reduced by selecting the location of the fibers such that back-scattering from one fiber is not detected by an adjacent fiber. The signals may also be separately used or pulsed, such that the reflected light is measured by only one fiber at a given time, or the wavelengths varied to distinguish one signal from another.

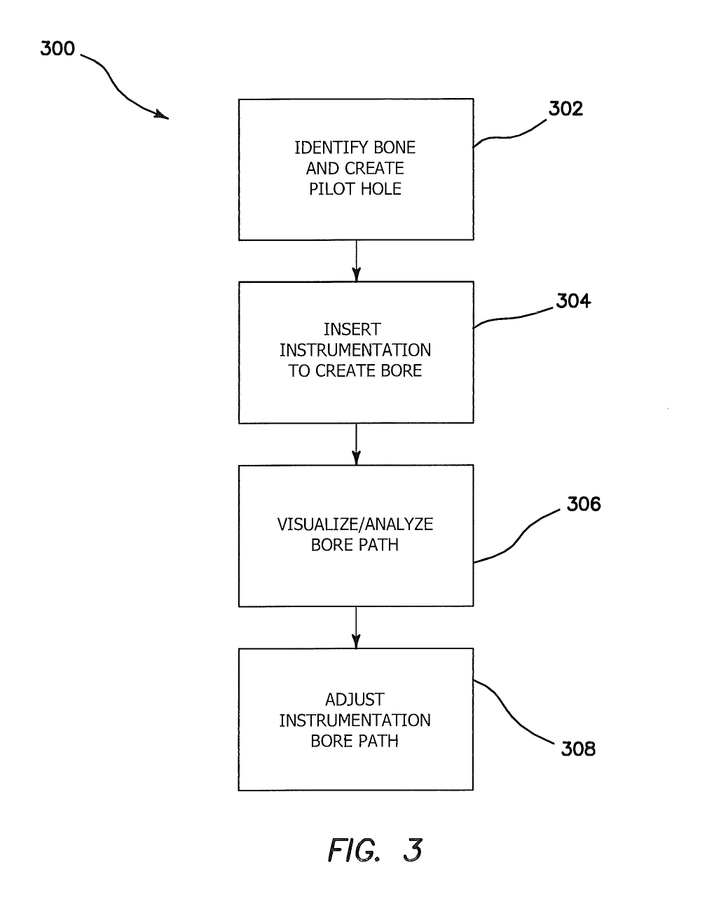

FIG. 3 illustrates a representative method 300 of using the OCT system 100 to navigate an orthopedic instrument through a bone sample. The exemplary method describes placement of a pedicle screw within a vertebrae. However, the invention is not so limited. Embodiments may be used with other orthopedic device to assist in navigation and placement in and around the bony structures of the body. It will be understood that no particular order for the steps in the methods described is required unless expressly stated and that some embodiments may use alternative orders for the steps, add, or omit certain steps.

In the exemplary embodiment of navigating around the spinal column, generally, block 302, a surgeon will expose the spine from the back and identify the target structure for placing the orthopedic device. For the exemplary embodiment of placing a pedicle screw, a pilot hole is created on the vertebrae at the insertion location of the stabilizing screw, at 204 of FIG. 2B. Under general anesthesia, a posterior midline incision may be made in the customary fashion, and muscles dissected to expose the spine. With a punch or tap, the drill entry site is marked.

At block 304, an orthopedic instrument including a bone drill is advanced to the starter hole. At low speed, the drill is used to create a bore in the bone. The bone drill creates a trajectory for the bone screw within the vertebrae without compromising the spinal cord or vertebral artery. During the drilling procedure at block 306, the trajectory immediately in front of the bone drill may be visualized using the OCT probe 104. The lateral substructures may also be viewed to ensure proper placement of the bore. From the image and statistical information provided by the OCT system, the trajectory of the bore may be maintained or altered, at block 308.

The OCT system may provide a visual indication of the bone and tissue immediately in front of the orthopedic device, thus assisting in navigation or trajectory of the device. The collected data may be converted to real-time video images or enhanced into computer generated images (CGI) or 3-dimentional (3-D) images to reconstruct the bony anatomy and its relations to surrounding soft tissue. The OCT probe may be used to detect the various densities of the bone, and use the different attributes to determine the location of the probe tip and the approaching structures. For example, the OCT probe may detect the cortical bone as an opaque, hard bony structure. Once inside the cancellous bone of the vertebrae, the OCT probe may detect the presence of the cancellous bone when the device is following the proper trajectory. If the trajectory approaches the spinal column or soft tissue and blood within the spinal canal, the image from the OCT probe will change to indicate the different material in the path of the probe. Therefore, a surgeon will be alerted of the approaching area to avoid before contact with the sensitive areas are encountered.

The OCT system may be used to analyze the signals received from the OCT probe to provide a visual or audile indication of the probe trajectory. For example, the OCT system may be used to display a real time image of the procedure, including the tissue structures in front of the probe tip. The system may determine that the trajectory of the probe is “safe” if the reflected signal indicates the same cancellous bone material remains in front of the probe. The system may also determine that a “danger zone,” including the spinal canal, is within the trajectory of the probe tip if the reflected signal indicates that the material change in front of the probe is of soft tissue compared to the bone material of the vertebrae. The system may provide feedback in a representative visual image of the trajectory, a visual alarm such as a color indicator, or as an audible sound, such as a beep, buzz, alarm, or other sensory signal.

The OCT system may provide other information to assist in the orthopedic procedure. For example, the OCT system may be used to take various measurements. The OCT system may provide sizing information for the bone screw or a distance to a danger zone. The OCT system may also include memory to retain snap-shots of the images during the procedure. For example, the system may be designed to retain a set number of images per time period, such as 10 images per second. During or after the procedure, the OCT system may compile these images into a three-dimension presentation of the bony structure or procedure. The OCT system may include computer graphics and rendering programs to use these images to present a three-dimensional image of the traversed structure.

What is claimed is:

1. A method of osteo-navigation, comprising:
identifying a target bone structure;
insert of an orthopedic instrument including an optical probe for analyzing a characteristic of the target bone structure into the target bone structure; and
navigating the orthopedic instrument into the target bone structure along a selected path in response to a signal from the optical probe.

2. The method of claim 1, further comprising creating a pilot hole at a desired location in the target bone structure once the target bone structure has been identified.

3. The method of claim 1, wherein the orthopedic instrument comprises a bone drill including the optical probe along a longitudinal axis of the bone drill capable of projecting optical radiation from a tip of the bone drill.

4. The method of claim 1, wherein the optical probe projects optical radiation from a tip of the bone drill and an adjacent side of the bone drill.

5. The method of claim 1, wherein the optical probe uses optical coherence tomography to create a visual image of a portion of the target bone structure.

6. The method of claim 1, wherein the signal from the optical probe comprises a visual representation of a portion of the target bone structure.

7. The method of claim 1, wherein the signal from the optical probe comprises an audible sound generated in response to a variation in a substructure of the target bone structure.

8. The method of claim 1, wherein navigating the orthopedic instrument into the target bone structure along the selected path in response to a signal from the optical probe, further comprises:
projecting optical radiation into the target bone structure;
capturing backscattered optical radiation reflected from a substructure of the target bone structure;
creating a visual representation of the captured backscattered optical radiation; and
advancing the orthopedic instrument further into the target bone structure along the selected path or altering the selected path of the orthopedic instrument in response to the visual representation.

9. The method of claim 8, wherein the optical probe is scanned laterally to create a two-dimensional image of the target bone structure.

10. The method of claim 8, wherein the visual representation of the captured backscattered optical radiation is compared to a control image to determine an approach of a substructure of interest.

11. The method of claim 10, wherein the substructure of interest is soft tissue.

12. A method of placing a pedicle screw in a vertebrae of a patient, comprising:
exposing a spine of the patient;
identifying and marking an entry site for the pedicle screw into the vertebrae;
advancing a bone drill including an optical probe into the vertebrae;
detecting a substructure of the vertebrae with the optical probe by optical coherence tomography in a path of the advancing bone drill; and
selecting a continued path of the bone drill based on the detected substructure of the vertebrae.

13. The method of claim 12, further comprising detecting a lateral substructure of the vertebrae flanking the path of the advancing bone drill.

14. The method of claim 12, further comprising analyzing a signal from the optical probe to provide statistical information of the substructure of the vertebrae.

15. The method of claim 14, wherein the statistical information is a distance to the substructure from the bone drill.

16. The method of claim 12, further comprising converting a signal from the optical coherence tomography to a real-time image of the vertebrae.

17. The method of claim 12, further comprising capturing repeated signals from the optical probe to create a reconstructed three-dimensional image of the vertebrae.

18. The method of claim 12, further comprising generating a warning signal when a danger zone is detected.

19. The method of claim 12, further comprising calculating a desired screw size based on information received by the optical probe.

20. An orthopedic surgical device for creating a bore in bone for placing a pedicle screw, comprising:
a handle;
a drill bit coupled to the handle capable of creating the bore in bone;
an optical probe including a fiber along a longitudinal axis of the drill bit, capable of projecting optical radiation from a tip of the drill bit, receiving backscattered radiation from a substructure in a trajectory of the drill bit, and creating an image of the substructure based on the received backscattered radiation; and
a display for the image of the substructure.
